(19) United States
(12) Patent Application Publication
(10) Pub. No.: US 2017/0258781 A1
(43) Pub. Date: Sep. 14, 2017

(54) ATRIAL SPECIFIC ANTIARRHYTHMICS FOR USE IN TREATING OR PREVENTING ATRIAL FIBRILLATION

(71) Applicant: Tufts Medical Center, Inc., Boston, MA (US)

(72) Inventors: Sami NOUJAIM, Land O Lakes, FL (US); Richard H. KARAS, Franklin, MA (US)

(21) Appl. No.: 15/532,179

(22) PCT Filed: Dec. 2, 2015
(86) PCT No.: PCT/US15/63457

§ 371 Date: Jun. 1, 2017

Related U.S. Application Data
(60) Provisional application No. 62/086,505, filed on Dec. 2, 2014.

(51) Int. Cl. A61K 31/4706; A61K 45/06

(52) U.S. Cl. CPC A61K 31/4706; A61K 45/06

(57) ABSTRACT

The invention relates to methods and compositions for treating or preventing atrial fibrillation.

The invention relates to methods and compositions for treating or preventing atrial fibrillation.

### BACKGROUND OF THE INVENTION

Atrial fibrillation (AF) is the most common heart rhythm disturbance. AF is associated with significant morbidity, and current pharmacologic treatment is inadequate. For example, depending on the study and the antiarrhythmic drug, the efficacy of AF termination with pharmacotherapy is 10 to 45% compared to placebo. In the U.S. alone, AF prevalence is projected to increase from 5 million cases in 2010 to 12 million cases in 2030. Therefore, there is an increasing need for the development of new treatments for AF.

The acetylcholine sensitive, inward-rectifier potassium channel (I_{KACh}) is mainly expressed in the atria and has been demonstrated to play a major role in AF perpetuation. Presently, the available clinical antiarrhythmic armamentarium lacks a selective I_{KACh} blocker.

### SUMMARY OF THE INVENTION

The invention provides methods of treating or preventing atrial fibrillation. The methods involve administering an effective amount of an atrial specific antiarrhythmic (e.g., a compound of formula (I), such as enantiopure (S)-chloroquine, compound A, or a pharmaceutically acceptable salt of one of such compounds) to a subject in need thereof. In some embodiments, the atrial fibrillation is permanent AF, persistent/chronic AF, or paroxysmal AF. In other embodiments, the atrial fibrillation is accompanied by atrial flutter. The atrial specific antiarrhythmic can be an inhibitor of I_{KACh}.

The methods of the invention can optionally include administering a second antiarrhythmic (e.g., amiodarone, dronedarone, quinidine, procainamide, disopyramide, encainide, flecainide, propafenone, moricizine, sotalol, dofetilide, and/or ibutilide) to the subject.

The methods of the invention can optionally include administering an anticoagulant (warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and/or dabigatran) to the subject.

In particular embodiments of the above methods, the atrial specific antiarrhythmic is a compound of formula (I):

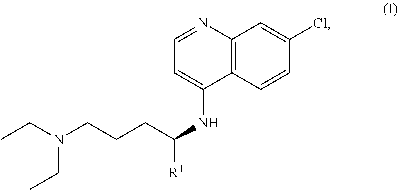

or a pharmaceutically acceptable salt thereof. In formula (I), R¹ is selected from optionally substituted C₁₋₆ alkyl and optionally substituted alkaryl. In certain embodiments R¹ is selected from methyl, ethyl, n-propyl, iso-propyl, cyclopropyl, benzyl, alpha-methylbenzyl, and phenethyl. The compound of formula (I) can be, e.g., selected from chloroquine or compound A (each depicted below), or a pharmaceutically acceptable salt thereof.

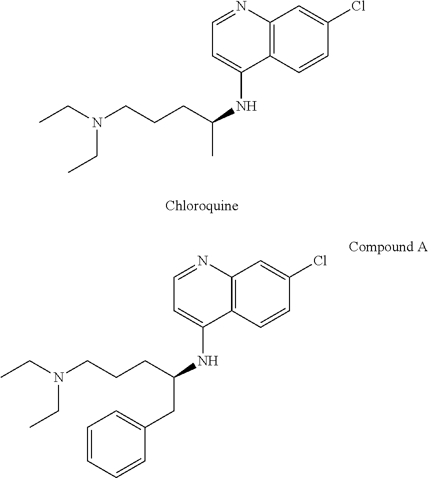 [SMILES: CCN(CC)CCC[C@H](C)Nc1ccnc2cc(Cl)ccc12 ; CCN(CC)CCC[C@H](Cc1ccccc1)Nc1ccnc2cc(Cl)ccc12]

The methods of the invention involve in some embodiments oral administration of 300-600 mg of a compound of formula (I), e.g., (S)-chloroquine, Compound A, or a pharmaceutically acceptable salt of one of these compounds.

The methods also include, optionally, treating or preventing atrial flutter associated with, or occurring concurrently with, atrial fibrillation.

The invention also includes pharmaceutical compositions including compounds of formula (I), such as enantiopure (S)-chloroquine, Compound A, or a pharmaceutically acceptable salt of one of these compounds and a pharmaceutically acceptable excipient. The pharmaceutical compositions can optionally include a second antiarrhythmic (e.g., amiodarone, dronedarone, quinidine, procainamide, disopyramide, encainide, flecainide, propafenone, moricizine, sotalol, dofetilide, and/or ibutilide). The pharmaceutical compositions can optionally include an anticoagulant (warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and/or dabigatran).

The invention further includes kits that include compounds of formula (I), such as an atrial specific antiarrhythmic (e.g., enantiopure (S)-chloroquine, Compound A, or a pharmaceutically acceptable salt of one of these compounds) and an anticoagulant (warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and dabigatran) and/or a second antiarrhythmic (quinidine, procainamide, disopyramide, encainide, flecainide, propafenone, moricizine, sotalol, and ibutilide).

In particular embodiments, the subject is diagnosed with AF prior to administration. In other embodiments, the subject is not suffering from comorbid rheumatic disease, malaria, systemic lupus, or Parkinson's disease.

The invention also includes use of the compounds described herein for preparing medicaments for the recited indications, or for use in treating or preventing the indications.

By “atrial specific antiarrhythmic” is meant a compound capable of inhibiting predominantly atrial arrhythmias with a relative lack of effect on ventricular electrophysiology. This specificity results from inhibiting predominantly I_{KACh}, which is restricted primarily to the atria, to a greater extent than I_{Kr}. In particular embodiments, the atrial specific antiarrhythmic blocks I_{KACh} to at least twice the extent of I_{Kr}, e.g., at least three times, at least four times, at least five times, or at least ten times.

A chloroquine preparation is “enantiopure” if at least 60% (e.g., at least 70, 80, 90, 95, 99, or 99.9%) of the chloroquine in the preparation is a particular enantiomer (e.g., (S)-chloroquine). Similarly, the term “enantiopure” is applies to the preparation of any compound of formula (I) or Compound A if at least 60% (e.g., at least 70, 80, 90, 95, 99, or 99.9%) the compound in the preparation is a particular enantiomer.

As used herein, the term “pharmaceutically acceptable salt” means any salt of an atrial specific antiarrhythmic, e.g., of a compound of formula (I), such as (S)-chloroquine or Compound A, within the scope of sound medical judgment, suitable for use in contact with the tissues of humans and animals without undue toxicity, irritation, allergic response and commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of (S)-chloroquine or separately by reacting a free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts.

As used herein, the term “subject” refers to any organism to which an atrial specific antiarrhythmic, such as a compound of formula (I) (e.g., (S)-chloroquine, Compound A, or a pharmaceutically acceptable salt of such a compound) may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be currently receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The term “prophylactic treatment,” as used herein, refers to treatment initiated, for example, prior to the onset of a disease, disorder, or condition, or prior to a potential recurrence thereof.

In the generic descriptions of compounds of this invention, the number of atoms of a particular type in a substituent group is generally given as a range, e.g., an alkyl group containing from 1 to 6 carbon atoms or C₁₋₆ alkyl. Reference to such a range is intended to include specific references to groups having each of the integer number of atoms within the specified range. For example, an alkyl group from 1 to 6 carbon atoms includes each of C₁, C₂, C₃, C₄, C₅, and C₆. As used herein, the terms “alkyl” and the prefix “alk-” are inclusive of both straight chain (e.g., methyl, ethyl, n-propyl, n-butyl, n-pentyl, and n-hexyl groups) and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups. The alkyl group may be substituted to include from 1 to 3 substituents, or can be unsubstituted. Exemplary substituents include hydroxy, alkoxy, aryloxy, sulfhydryl, alkylthio, arylthio, halide, fluoroalkyl, carboxyl, hydroxyalkyl, carboxyalkyl, amino, aminoalkyl, mono substituted amino, disubstituted amino, and quaternary amino groups.

By “alkaryl” is meant an alkyl substituted by an aryl group having from 7 to 14 carbon atoms. By “aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The alkaryl group may be substituted to include from 1 to 3 substituents, or can be unsubstituted. Exemplary substituents include methyl, hydroxy, alkoxy, aryloxy, sulfhydryl, alkylthio, arylthio, halide, fluoroalkyl, carboxyl, hydroxyalkyl, carboxyalkyl, amino, aminoalkyl, mono substituted amino, disubstituted amino, and quaternary amino groups. Alkaryl groups include, e.g., benzyl, phenethyl, alpha-methylbenzyl, and 3,4-dichlorophenethyl.

### BRIEF DESCRIPTION OF THE DRAWINGS

FIG. 1: In patch clamp experiments, I_{KACh} was measured in human embryonic kidney cells (HEK 293) transfected with I_{KACh}. At 1 μm, racemic chloroquine blocked 42% of the outward component of I_{KACh}. (S)-chloroquine blocked more than 83%, and (R)-chloroquine less than 13%.

FIG. 2: I_{Kr} current was measured in Chinese hamster ovary cells (CHO) expressing the current. At 1 μm, racemic chloroquine blocked 33% of the I_{Kr} tail current. (S)-chloroquine blocked 16%, and (R)-chloroquine less than 32%.

FIG. 3: Representative traces of I_{KACh} block with racemic chloroquine and its enantiomers in patch clamp experiments. The boxed area is the physiologically important outward component of the I_{KACh} current. A magnification is shown. Currents are elicited by 2 second voltage ramps from −130 mV to +50 mV from a holding potential of −40 mV. The traces are the barium sensitive currents.

FIG. 4: Representative traces of I_{Kr} block with racemic chloroquine and its enantiomers in patch clamp experiments. The arrow denotes the tail current. Tail current is elicited by 3 second step voltage drop from +20 mV to −40 mV. The gray trace is the remaining current after total current block with E4031.

FIG. 5: K_{KACh} and I_{Kr} were measured in cells expressing the currents, as described above, in the presence of chloroquine racemate or Compound A. Percentage block of the indicated currents is shown.

### DETAILED DESCRIPTION OF THE INVENTION

The invention provides methods and compositions for treating or preventing AF in subjects using an atrial specific antiarrhythmic, e.g., an agent that inhibits I_{KACh}. The use of an atrial specific antiarrhythmic provides reduced risk of side effects, in particular, ventricular proarrhythmia. Antiarrhythmics, in general, carry a risk of proarrhythmia due to I_{Kr} blockade that leads to significant QT prolongation and the resulting occurrence of torsades de points and other forms of ventricular tachycardia. We have identified (S)-chloroquine and Compound A as specific inhibitors of I_{KACh} and, accordingly, as atrial specific antiarrhythmics.

In humans with AF and in animal models of chronic AF, I_{KACh} is remodeled. Without being bound by theory, I_{KACh} is a current that flows through tetrameric sarcolemmal channels formed by Kir3.1 and Kir3.4 proteins. In baseline physiology, I_{KACh} activity is minimal. Upon parasympathetic stimulation, acetylcholine binds to the G-protein coupled muscarinic (M2) receptor leading to channel activation. Since I_{KACh} is important in heart rate modulation, the on/off switching of the current is a tightly regulated process. However, in AF, I_{KACh} is constitutively active, irrespective of parasympathetic stimulation. This leads to a net increase in the background inward rectifier current. We have shown that increasing the inward rectifier current leads to shortening of the APD, and the subsequent formation of stable electrical rotors which activate the myocardium at high frequencies, and thus lead to fibrillation.

Chloroquine is a quinoline that has been used extensively in the clinic as an antimalarial. Chloroquine is a racemic mixture. The enantiomers of chloroquine can be separated from the racemic mixture using HPLC (Davos Pharma) or other techniques. While stereoselectivity has been demonstrated against some malarial strains (Ducharme J, Farinotti R. Clinical pharmacokinetics and metabolism of chloroquine. Focus on recent advancements. Clinical pharmacokinetics. 1996; 31:257-274; Brocks D R, Mehvar R. Stereoselectivity in the pharmacodynamics and pharmacokinetics of the chiral antimalarial drugs. Clinical pharmacokinetics. 2003; 42:1359-1382), and the (R)-enantiomer of chloroquine has been suggested for use in treating Parkinson's disease (U.S. Pat. No. 6,417,177), the use of (S)-chloroquine as an I_{KACh} inhibitor and subsequently as a treatment for AF is novel and has not been attempted before.

We have discovered that (S)-chloroquine is a significantly greater blocker of I_{KACh}, relative to racemic or (R)-chloroquine. In addition, (S)-chloroquine is a much weaker blocker of I_{Kr}, relative to racemic or (R)-chloroquine. Thus, (S)-chloroquine surprisingly has increased activity relative to racemic or (R)-chloroquine and, in having specificity for I_{KACh}, provides an additional level of safety with respect to proarrythmic side effects of known treatments. We have also found that Compound A has improved properties relative to racemic chloroquine.

Other atrial specific antiarrhythmics can be identified using the techniques described herein.

### Atrial Specific Antiarrhythmics

The atrial specific antiarrhythmics of the invention can include compounds of formula (I):

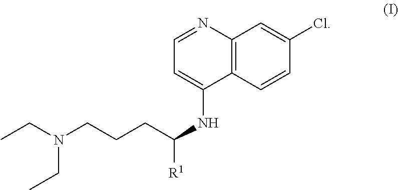

In formula (I), R¹ is selected from optionally substituted C₁₋₆ alkyl and optionally substituted alkaryl. In certain embodiments R¹ is selected from methyl, ethyl, n-propyl, iso-propyl, benzyl, alpha-methylbenzyl, and phenethyl.

Compounds of formula (I) can be synthesized using methods analogous to those described in U.S. Pat. Nos. 5,596,002; 5,948,791; and 4,421,920, each of which is incorporated herein by reference. Additional synthetic techniques that can be useful in the synthesis of compound of formula (I) are described in the art (see, e.g., Solomon et al. Journal of medicinal chemistry 50:394 (2007); Solomon et al., Bioorganic & medicinal chemistry 13:2157 (2005); and Schmidt et al., Antimicrobial agents and chemotherapy 11:826 (1977)).

Enantiopure formulations of the compounds of formula (I) can be prepared using enantioselective syntheses, or by separating a mixture enantiomers (e.g., using enantioselective chromatography or crystallization techniques) to produce an enantiopure composition.

### Utility and Administration

The invention provides methods of treating or preventing AF in a subject by administering to the subject an atrial specific antiarrhythmic, e.g., a compound of formula (I), such as (S)-chloroquine, Compound A, or a pharmaceutically acceptable salt of one of these compounds (or a pharmaceutical composition described herein). The methods of the invention may be employed to treat or prevent permanent, persistent/chronic, or paroxysmal AF. Optionally, a treated subject may have concurrent atrial flutter that is treated or prevented by the methods of the invention.

Treatment according to the methods of the invention can be preventative or prophylactic (e.g., to prevent AF in patients (e.g., at risk patients) who have not experienced AF, as well as to prevent further AF in patients who have experienced one or more episodes of AF in the past, and are at risk of future AF episodes) or therapeutic (e.g., to treat patients currently experiencing AF).

For use in the treatment of human and animal subjects, the atrial specific antiarrhythmic, e.g., a compound of formula (I), (S)-chloroquine, Compound A, or a pharmaceutically acceptable salt of one of these compounds, can be formulated in a pharmaceutical or veterinary composition. Depending on the subject to be treated, the mode of administration, and the type of treatment desired (e.g., prevention, prophylaxis, or therapy) the atrial specific antiarrhythmic, e.g., a compound of formula (I), (S)-chloroquine, Compound A, or a pharmaceutically acceptable salt of one of these compounds, is formulated in a way consonant with these parameters. A summary of such techniques is found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York.

The atrial specific antiarrhythmic, e.g., a compound of formula (I), (S)-chloroquine, Compound A, or a pharmaceutically acceptable salt of one of these compounds, may be present in amounts totaling 1-95% by weight of the total weight of a composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, gastrointestinal, reproductive or oral mucosa, or targeted tissue delivery. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

The atrial specific antiarrhythmic, e.g., a compound of formula (I), (S)-chloroquine, Compound A, or a pharmaceutically acceptable salt of one of these compounds, may be prepared and used as pharmaceutical compositions including an effective amount of a atrial specific antiarrhythmic, e.g., a compound of formula (I), (S)-chloroquine, Compound A, or a pharmaceutically acceptable salt of one of these compounds, and a pharmaceutically acceptable carrier or excipient, as is well known in the art. In some embodiments, the composition includes at least two different pharmaceutically acceptable excipients or carriers.

Formulations may be prepared in a manner suitable for systemic administration or topical or local administration. Systemic formulations include those designed for injection (e.g., intramuscular, intravenous, or subcutaneous injection) or may be prepared for transdermal, transmucosal, or oral administration. The formulation will generally include a diluent as well as, in some cases, adjuvants, buffers, and/or preservatives. The atrial specific antiarrhythmic, e.g., a compound of formula (I), (S)-chloroquine, Compound A, or a pharmaceutically acceptable salt of one of these compounds, can also be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, and glycerol. Such compositions may also contain amounts of nontoxic auxiliary substances such as wetting or emulsifying agents, and pH buffering agents, such as, for example, sodium acetate, sorbitan monolaurate, and so forth. Various sustained release systems for drugs have also been devised. See, for example, U.S. Pat. No. 5,624,677.

Formulations for oral use include tablets containing the active ingredient(s) in a mixture with non-toxic pharmaceutically acceptable excipients. These excipients include, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, and buffering agents.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the atrial specific antiarrhythmic, e.g., a compound of formula (I), (S)-chloroquine, Compound A, or a pharmaceutically acceptable salt of one of these compounds, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the dosage of any of the atrial specific antiarrhythmic, e.g., a compound of formula (I), (S)-chloroquine, Compound A, or a pharmaceutically acceptable salt of one of these compounds, will depend on the nature of the compound, and can readily be determined by one skilled in the art. Typically, such dosage is normally about 0.001 mg to 2000 mg per day, desirably about 1 mg to 1000 mg per day, and more desirably about 5 mg to 500 mg per day (e.g., 150-300 mg).

The dose of the atrial specific antiarrhythmic, e.g., a compound of formula (I), (S)-chloroquine, Compound A, or a pharmaceutically acceptable salt of one of these compounds, depends on a number of factors, such as, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount.” The administration can be conducted in a single unit dosage form with continuous therapy or in a single dose therapy ad libitum.

### Combination Therapies

In some embodiments, a pharmaceutical composition or method of use of the invention may further include one or more additional compounds, e.g., an antiarrhythmic and/or an anticoagulant and/or upstream therapies. Examples of antiarrhythmics include amiodarone, dronedarone, quinidine, procainamide, disopyramide, encainide, flecainide, propafenone, moricizine, sotalol, dofetilide, and ibutilide. Examples of anticoagulants include warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and dabigatran. Examples of upstream therapies include benazepril, captropil, enalapril, fosinopril, lisinopril, moexipril, perindopril, quinapril, ramipril, trandolapril, azilsartan, candesartan, eprosartan, irbesartan, losartan, olmesartan, telmisartan, valsartan, acebutolol, atenolol, bisoprolol, metoprolol, nadolol, and propranolol.

It will also be appreciated that a compound of formula (I), such as (S)-chloroquine or Compound A (or a pharmaceutically salt thereof) and pharmaceutical compositions of the present invention can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

Depending on the mode of administration, the compounds will be formulated into suitable compositions to permit facile delivery. Each compound of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

The individually or separately formulated agents can be packaged together as a kit. Non-limiting examples include, but are not limited to, kits that contain, e.g., two pills, a pill and a powder, a suppository and a liquid in a vial, or two topical creams. The kit can include optional components that aid in the administration of the unit dose to subjects, such as vials for reconstituting powder forms, syringes for injection, customized IV delivery systems, or inhalers. Additionally, the unit dose kit can contain instructions for preparation and administration of the compositions. The kit may be manufactured as a single use unit dose for one subject, multiple uses for a particular subject (at a constant dose or in which the individual compounds may vary in potency as therapy progresses); or the kit may contain multiple doses suitable for administration to multiple subjects (“bulk packaging”). The kit components may be assembled in cartons, blister packs, bottles, and tubes.

Two or more compounds may be mixed together in a tablet, capsule, or other vehicle or may be partitioned. In one example, the first compound is contained on the inside of the tablet, and the second compound is on the outside, such that a substantial portion of the second compound is released prior to the release of the first compound.

Administration of each drug in a combination therapy, as described herein, can, independently, be one to four times daily for one day to one year, and may even be for the life of the subject. Chronic, long-term administration may be indicated.

### Examples

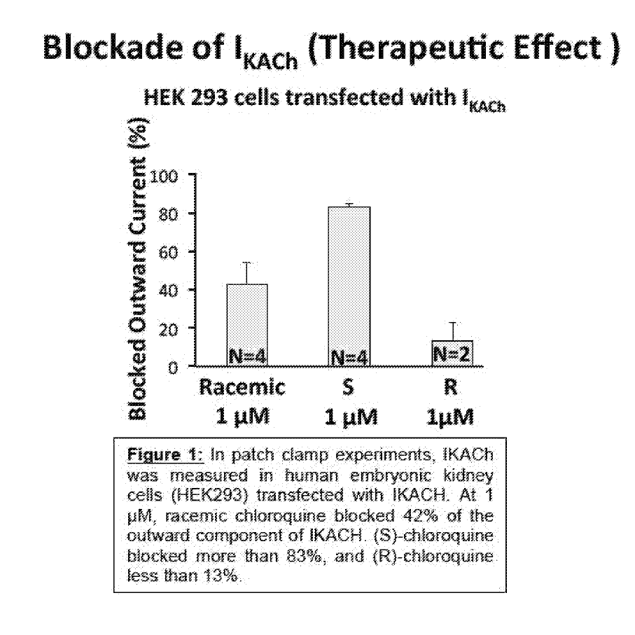

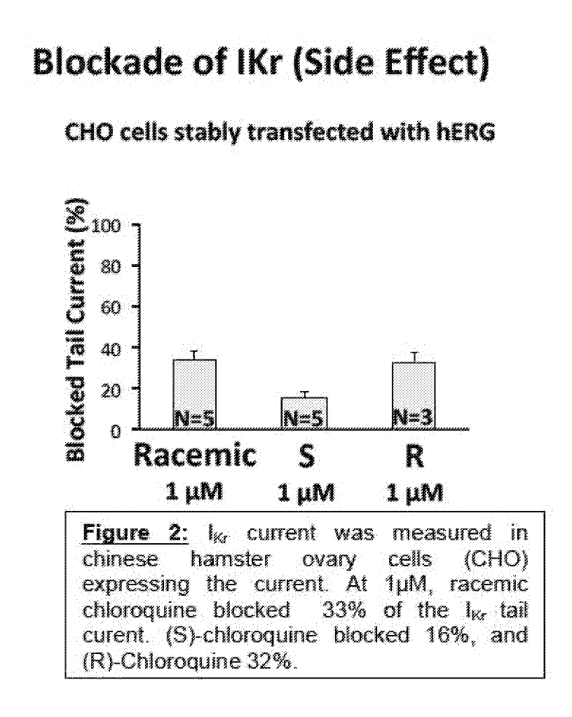

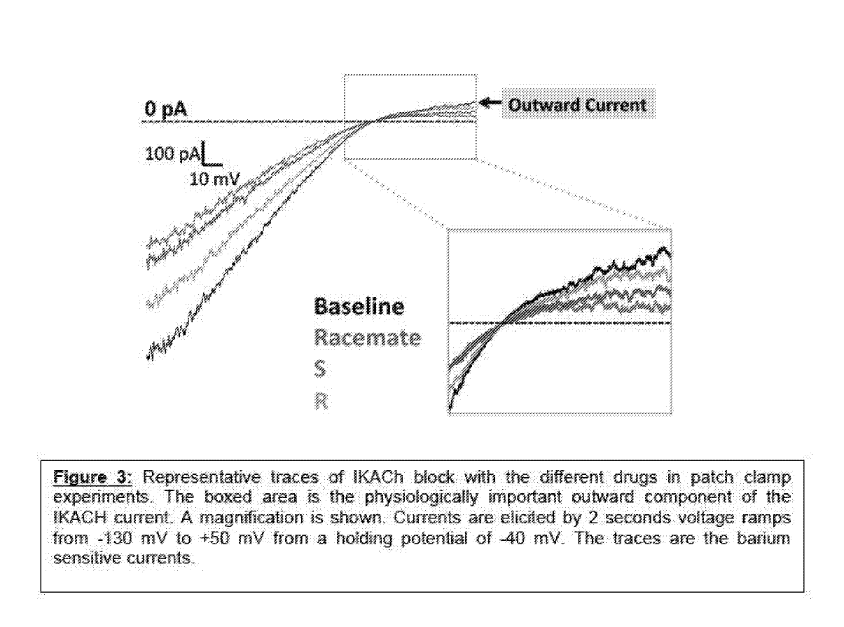

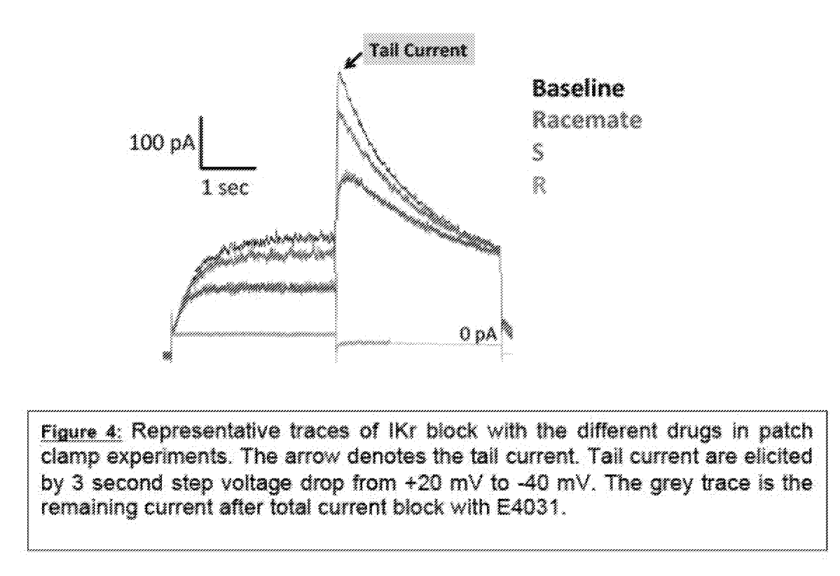

At 1 μM, racemic chloroquine blocked 42%±11 of the I_{KACh} current, while the eutomer (S) blocked 83%±2 and the distomer (R) blocked 14%±9 (FIG. 1). Additionally, the eutomer had less cardiac toxicity (block of the delayed rectifier current I_{Kr}) since at 1 μM it blocked 16%±2.5 of I_{Kr}. Racemic chloroquine and the distomer block 32.9%±1.2 and 32.7%±4.7 of I_{Kr}, respectively (FIG. 2). FIGS. 3 and 4 are representative current traces of I_{KACh} and I_{Kr}, respectively. They show that while the S enantiomer is the best blocker of I_{KACh}, it is the weakest blocker of I_{Kr}, as compared to the R enantiomer and the racemic drug.

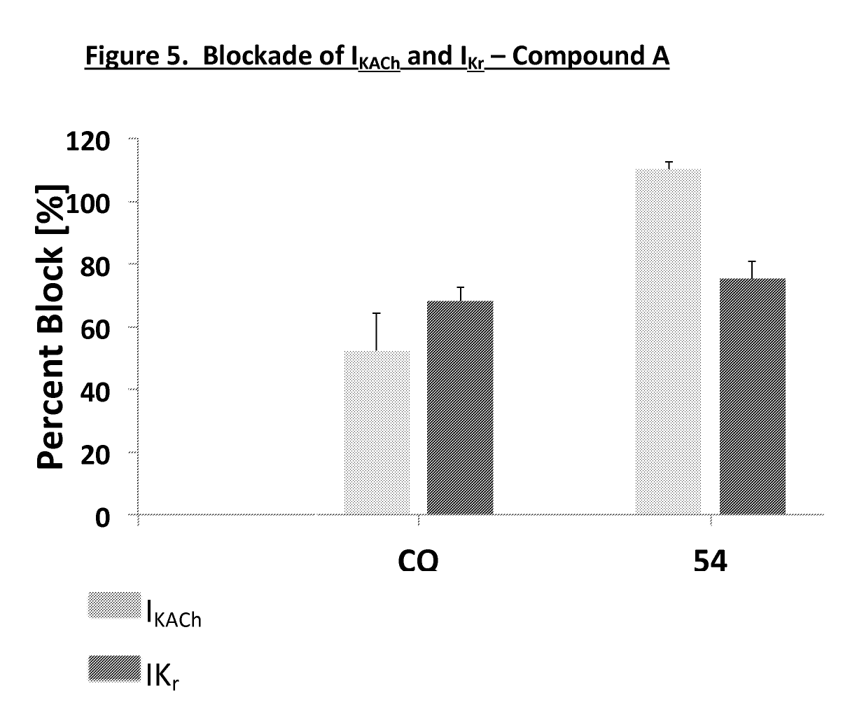

In other experiments, Compound A was tested for block of I_{KACh} and I_{Kr}, as compared to racemic chloroquine in the same manner, and in the same cell lines, as was done with S-chloroquine and R-chloroquine. As shown in FIG. 5, Compound A blocked 110%±2.5 (n=4 cells) of the I_{KACh} current, while chloroquine blocked 52%±11 (n=5). Furthermore, Compound A blocked 75%±5.7 (n=5) of the I_{Kr} current, while chloroquine blocked 68%±2.8 9 (n=5). Thus, Compound A blocks I_{Kr} to the same extent as chloroquine, but is a significantly better blocker of I_{KACh}.

We used the conventional patch clamp method and human embryonic kidney cells transfected with Kir3.1 and Kir3.4 protein channels responsible for I_{KACh}, or Chinese hamster ovary cells transfected with the hERG protein channel responsible for I_{Kr}. The currents were measured in single cells and the effects of 1 μM (S) or (R) or racemic chloroquine were tested. In order to compute the percentage of blocked currents, at the end of each experiment, I_{KACh} was fully blocked with 1 mM barium and I_{Kr} was blocked with 10 μM E4031. In order to elicit I_{KACh}, the cells were subjected to a voltage protocol consisting of a 2 second ramp from −130 mV to 50 mV, from a holding potential of −40 mV. I_{Kr} tail current was elicited by a 3 second voltage drop step from 20 mV to −40 mV. The drugs were superfused over a 5 minute period into the chambers that hold the cells. Percentage of blocked current was taken as:

$$\frac{( initial current - current after chloroquine )}{( initial current - current after Ba or E 4031 )} \times 100$$

The invention further encompasses the subject matter described in the following numbered paragraphs.
1. A method of treating or preventing atrial fibrillation (AF) comprising administering an effective amount of an atrial specific antiarrhythmic to a subject in need thereof.
2. The method of paragraph 1, wherein said atrial fibrillation is accompanied by atrial flutter.
3. The method of paragraph 1 or 2, wherein the atrial specific antiarrhythmic is an inhibitor of I_{KACh}.
4. The method of any one of paragraphs 1-3, wherein the AF is permanent AF.
5. The method of any one of paragraphs 1-3, wherein the AF is persistent/chronic AF.
6. The method of any one of paragraphs 1-3, wherein the AF is paroxysmal AF.
7. The method of any one of paragraphs 1-6, further comprising administering a second antiarrhythmic to the subject.
8. The method of paragraph 7, wherein the second antiarrhythmic is selected from the group consisting of amiodarone, dronedarone, quinidine, procainamide, disopyramide, encainide, flecainide, propafenone, moricizine, sotalol, dofetilide, and ibutilide.
9. The method of any one of paragraphs 1-8, further comprising administering an anticoagulant to the subject.
10. The method of paragraph 9, wherein the anticoagulant is selected from the group consisting of warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and dabigatran.
11. The method of any one of paragraphs 1-10, wherein the atrial specific antiarrhythmic is a compound of formula (I):

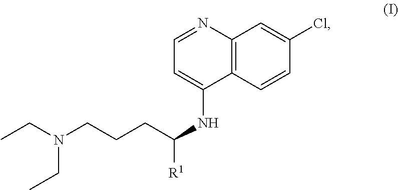

or a pharmaceutically acceptable salt thereof,
wherein R¹ is selected from optionally substituted C₁₋₆ alkyl and optionally substituted alkaryl.
12. The method of paragraph 10, wherein R¹ is selected from methyl, ethyl, n-propyl, iso-propyl, cyclopropyl, benzyl, alpha-methylbenzyl, and phenethyl.
13. The method of paragraph 10, wherein the atrial specific antiarrhythmic is compound A or a pharmaceutically acceptable salt thereof.
14. The method of paragraph 10, wherein the atrial specific antiarrhythmic is enantiopure (S)-chloroquine or a pharmaceutically acceptable salt thereof.
15. The method of paragraph 14, comprising administration of 150-300 mg of said enantiopure (S)-chloroquine or said pharmaceutically acceptable salt thereof.
16. The method of any one of paragraphs 1-15, wherein said administration is oral or parenteral.
17. The method of paragraph 16, wherein said parenteral administration is intravenous, subcutaneous, or intramuscular.
18. The method of any one of paragraphs 1-17, further comprising treatment of atrial flutter associated with, or occurring concurrently with, said atrial fibrillation.
19. A pharmaceutical composition comprising an enantiopure preparation of a compound of formula (I):

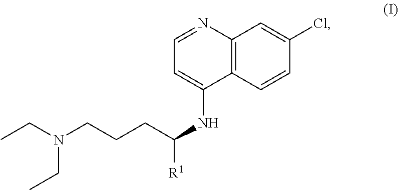

or a pharmaceutically acceptable salt thereof and a pharmaceutically acceptable excipient,
wherein R¹ is selected from optionally substituted C₁₋₆ alkyl and optionally substituted alkaryl.
20. The pharmaceutical composition of paragraph 19, wherein R¹ is selected from methyl, ethyl, n-propyl, iso-propyl, cyclopropyl, benzyl, alpha-methylbenzyl, and phenethyl.
21. The pharmaceutical composition of paragraph 20, wherein the compound of formula (I) is compound A or a pharmaceutically acceptable salt thereof.
22. The pharmaceutical composition of paragraph 20, wherein the compound of formula (I) is (S)-chloroquine or a pharmaceutically acceptable salt thereof.
23. The pharmaceutical composition of any one of paragraphs 19-22, further comprising a second antiarrhythmic.
24. The pharmaceutical composition of paragraph 23, wherein the second antiarrhythmic is selected from the group consisting of amiodarone, dronedarone, quinidine, procainamide, disopyramide, encainide, flecainide, propafenone, moricizine, sotalol, dofetilide, and ibutilide.
25. The pharmaceutical composition of any one of paragraphs 19-24, further comprising an anticoagulant.
26. The pharmaceutical composition of paragraph 25, wherein the anticoagulant is selected from the group consisting of warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and dabigatran.
27. A kit comprising an atrial specific antiarrhythmic and an anticoagulant or a second antiarrhythmic.
28. The kit of paragraph 27, further comprising a second antiarrhythmic.
29. The kit of paragraph 28, wherein the second antiarrhythmic is selected from the group consisting of quinidine, procainamide, disopyramide, encainide, flecainide, propafenone, moricizine, sotalol, and ibutilide.
30. The kit of any one of paragraphs 27-29, further comprising an anticoagulant.
31. The kit of paragraph 30, wherein the anticoagulant is selected from the group consisting of warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and dabigatran.
32. The kit of any one of paragraphs 27-31, wherein the atrial specific antiarrhythmic is a compound of formula (I):

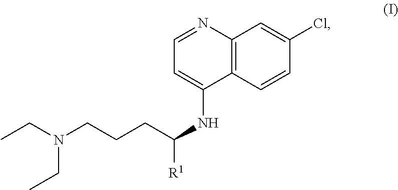

or a pharmaceutically acceptable salt thereof,
wherein R¹ is selected from optionally substituted C₁₋₆ alkyl and optionally substituted alkaryl.
33. The kit of paragraph 32, wherein R¹ is selected from methyl, ethyl, n-propyl, iso-propyl, cyclopropyl, benzyl, alpha-methylbenzyl, and phenethyl.
34. The kit of paragraph 32, wherein the atrial specific antiarrhythmic is compound A or a pharmaceutically acceptable salt thereof.
35. The kit of paragraph 32, wherein the atrial specific antiarrhythmic is enantiopure (S)-chloroquine or a pharmaceutically acceptable salt thereof.

1. A method of treating or preventing atrial fibrillation (AF) comprising administering an effective amount of an atrial specific antiarrhythmic to a subject in need thereof.

2. The method of claim 1, wherein said atrial fibrillation is accompanied by atrial flutter.

3. The method of claim 1, wherein the atrial specific antiarrhythmic is an inhibitor of I_{KACh}.

4. The method of claim 1, wherein the AF is permanent AF, persistent/chronic AF, or paroxysmal AF.

5. (canceled)

6. (canceled)

7. The method of claim 1, further comprising administering (a) a second antiarrhythmic to the subject, wherein optionally the second antiarrhythmic is selected from the group consisting of amiodarone, dronedarone, quinidine, procainamide, disopyramide, encainide, flecainide, propafenone, moricizine, sotalol, dofetilide, and ibutilide, or (b) an anticoagulant to the subject, wherein optionally the anticoagulant is selected from the group consisting of warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and dabigatran.

8-10. (canceled)

11. The method of claim 1, wherein the atrial specific antiarrhythmic is a compound of formula (I): 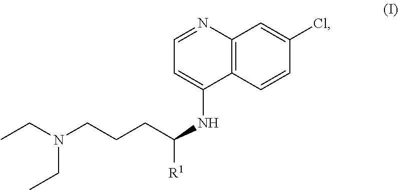
or a pharmaceutically acceptable salt thereof,
wherein R¹ is selected from optionally substituted C₁₋₆ alkyl and optionally substituted alkaryl.

12. The method of claim 11, wherein R¹ is selected from methyl, ethyl, n-propyl, iso-propyl, cyclopropyl, benzyl, alpha-methylbenzyl, and phenethyl.

13. The method of claim 11, wherein the atrial specific antiarrhythmic is compound A or a pharmaceutically acceptable salt thereof.

14. The method of claim 11, wherein the atrial specific antiarrhythmic is enantiopure (S)-chloroquine or a pharmaceutically acceptable salt thereof, and optionally 150-300 mg of said enantiopure (S)-chloroquine or said pharmaceutically acceptable salt thereof is administered.

15-17. (canceled)

18. The method of claim 1, further comprising treatment of atrial flutter associated with, or occurring concurrently with, said atrial fibrillation.

19. A pharmaceutical composition comprising an enantiopure preparation of a compound of formula (I): 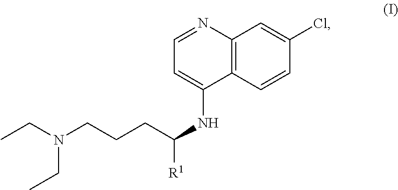
or a pharmaceutically acceptable salt thereof and a pharmaceutically acceptable excipient,
wherein R¹ is selected from optionally substituted C₁₋₆ alkyl and optionally substituted alkaryl.

20. The pharmaceutical composition of claim 19, wherein R¹ is selected from methyl, ethyl, n-propyl, iso-propyl, cyclopropyl, benzyl, alpha-methylbenzyl, and phenethyl.

21. The pharmaceutical composition of claim 20, wherein the compound of formula (I) is compound A or a pharmaceutically acceptable salt thereof.

22. The pharmaceutical composition of claim 20, wherein the compound of formula (I) is (S)-chloroquine or a pharmaceutically acceptable salt thereof.

23. The pharmaceutical composition of claim 19, further comprising (a) a second antiarrhythmic, wherein optionally the second antiarrhythmic is selected from the group consisting of amiodarone, dronedarone, quinidine, procainamide, disopyramide, encainide, flecainide, propafenone, moricizine, sotalol, dofetilide, and ibutilide, or (b) an anticoagulant, wherein optionally the anticoagulant is selected from the group consisting of warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and dabigatran.

24-26. (canceled)

27. A kit comprising an atrial specific antiarrhythmic and an anticoagulant or a second antiarrhythmic.

28. The kit of claim 27, further comprising (a) a second antiarrhythmic, wherein optionally the second antiarrhythmic is selected from the group consisting of amiodarone, dronedarone, quinidine, procainamide, disopyramide, encainide, flecainide, propafenone, moricizine, sotalol, dofetilide, and ibutilide, or (b) an anticoagulant, wherein optionally the anticoagulant is selected from the group consisting of warfarin, acenocoumarol, phenprocoumon, atromentin, brodifacoum, phenindione, heparin, low molecular weight heparin, fondaparinux, idraparinux, rivaroxaban, apixaban, edoxaban, hirudin, lepirudin, bivalirudin, argatroban, and dabigatran.

29-31. (canceled)

32. The kit of claim 27, wherein the atrial specific antiarrhythmic is a compound of formula (I): 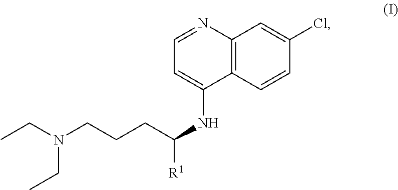
or a pharmaceutically acceptable salt thereof,
wherein R¹ is selected from optionally substituted C₁₋₆ alkyl and optionally substituted alkaryl.

33. The kit of claim 32, wherein R¹ is selected from methyl, ethyl, n-propyl, iso-propyl, cyclopropyl, benzyl, alpha-methylbenzyl, and phenethyl.

34. The kit of claim 32, wherein the atrial specific antiarrhythmic is compound A or a pharmaceutically acceptable salt thereof, or enantiopure (S)-chloroquine or a pharmaceutically acceptable salt thereof.

35. (canceled)
